(19) United States
(12) Plant Patent Application Publication
(10) Pub. No.: US 2016/0014946 P1
(43) Pub. Date: Jan. 14, 2016

(54) Dianthus Plant Named 'KLEDG14177'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/545,532

(22) Filed: May 18, 2015

Related U.S. Application Data
(60) Provisional application No. 61/998,888, filed on Jul. 10, 2014.

(51) Int. Cl. A01H 5/00

(52) U.S. Cl. PLT/272

(57) ABSTRACT

A new dianthus plant named ‘KLEDG14177’ particularly distinguished by dark purple flowers with a dark center, small, dark green to blue leaves, and very good branching, is disclosed.

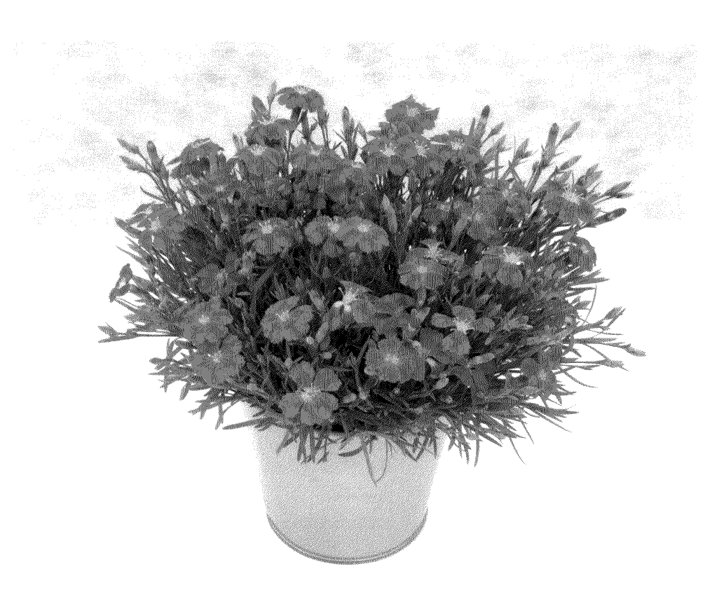

### CROSS REFERENCE TO RELATED APPLICATION

This application claims the benefit of U.S. Provisional Application No. 61/998,888 filed Jul. 10, 2014, the entire contents of which are incorporated herein by reference for all purposes.

### GENUS AND SPECIES

Dianthus sp.

### VARIETY DENOMINATION

‘KLEDG14177’

### BACKGROUND

The present invention comprises a new and distinct variety of Dianthus, botanically known as Dianthus sp. and hereinafter referred to by the variety name ‘KLEDG14177’. ‘KLEDG14177’ is a selection that resulted from a cross pollination of the proprietary female dianthus line ‘DSS 2009 0513’ (unpatented) and the male dianthus line ‘DSS 2008 0003’ (unpatented).

In August 2010 the proprietary female dianthus line ‘DSS 2009 0513’ and the proprietary male dianthus line ‘DSS 2008 0003’ were cross pollinated in Stuttgart, Germany and seeds were obtained. The seeds were sown and plants were grown for evaluation. A plant line was selected in June 2011 in Stuttgart, Germany, and named ‘KLEDG14177’. In June 2011, ‘KLEDG14177’ was first vegetatively propagated by vegetative cuttings. ‘KLEDG14177’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. Dark purple flowers with a dark center;

2. Small, dark green to blue leaves; and

3. Very good branching.

### DESCRIPTION OF THE PHOTOGRAPH

This new Dianthus plant is illustrated by the accompanying photograph, which shows the overall plant habit of the variety ‘KLEDG14177’ grown in a pot. The photograph is of a 10 to 12 week old plant in Stuttgart, Germany under greenhouse conditions in May 2013. The colors shown are as true as can be reasonably obtained by conventional photographic procedures.

### DESCRIPTION OF THE NEW VARIETY

The following detailed description set forth the distinctive characteristics of ‘KLEDG14177’. The data which define these characteristics were collected from asexual reproductions carried out in Stuttgart, Germany. Data was collected on four month old plants grown in a glass greenhouse in Stuttgart, Germany in March, 2014. Color references are to The R.H.S. Colour Chart of The Royal Horticultural Society of London (R.H.S.), 5^{th} edition.
Classification:
   Family.—Caryophyllaceae.
   Species.—Dianthus sp.
   Common name.—Garden carnation.
   Parentage.—Female: Proprietary dianthus line ‘DSS 2009 0513’ Male: Proprietary dianthus line ‘DSS 2008 0003’.
Propagation:
   Type.—Vegetative cuttings.
   Time to produce a rooted young plant.—About 20 days.
   Root description.—White, small, well-branched.
   Rooting habit.—Lanceolate.
Plant description:
   Plant form.—Bushy, potted and garden plant.
   Plant height.—10.0 cm.
   Plant diameter or spread.—18.0 cm.
Lateral branches:
   Length.—6.0 cm.
   Diameter.—1.5 cm.
   Internode length.—0.6 cm.
   Node length.—0.3 cm.
   Node diameter.—0.3 cm.
   Texture.—Smooth.
   Color.—RHS 137B.
Foliage description:
   Arrangement.—Opposite.
   Length.—5.0 cm.
   Width.—0.2 cm.
   Shape.—Lanceolate.
   Apex.—Acute.
   Base.—Obtuse.
   Margin.—Entire.
   Texture.—Upper and lower surfaces: Smooth.
   Color.—Immature and mature leaves: Upper surface: RHS 137B Lower surface: RHS 137C.
   Venation pattern.—Pinnate.
   Venation.—RHS 137D.
Flower description:
   Flower type and habit.—Single, round.
   Fragrance.—Absent.
   Natural flowering season.—Spring to autumn.
   Flower longevity.—6 to 8 days.
   Flower buds.—Length: 1.8 cm Diameter: 0.5 cm Shape: Tall cylinder to cylindrical Color: RHS 137C.
   Corolla.—Quantity of petals/arrangement: 5, round Corolla height: 0.5 cm Petal length: 3.0 cm. Petal width: 1.4 cm Petal shape: V-shaped Petal apex: Obtuse Petal base: Cuspidate Petal margin: Serrate. Petal texture: Upper and lower surfaces: Smooth. Petal color: Immature: Upper surface: RHS N74A and RHS 64A Lower surface: RHS N74C Mature: Upper surface: RHS N74A and RHS 61A Lower surface: RHS N74C Calyx: Quantity of sepals/arrangement: 5, single Calyx height: 2.0 cm Calyx diameter, at the apex: 0.5 cm Sepal texture: Upper and lower surfaces: Smooth Sepal color: Upper surface: RHS 137C Lower surface: RHS 137D.
Peduncles:
   Length.—6.5 cm.
   Diameter.—1.0 cm.
   Strength.—Moderate.
   Texture.—Smooth.
   Color.—RHS 136B.
Reproductive organs:
   Stamens.—Quantity: Greater than 5 Filament length: 1.0 cm Filament color: RHS 75C Anther length: 0.15 cm Anther diameter: 0.08 cm Anther color: RHS 75B Pollen amount: Moderate.
   Pistils.—Quantity: 1 Pistil length: 0.8 cm Style length: 0.1 cm Style color: RHS 76D Stigma shape: Filamentous Stigma color: RHS 72A Ovary size: 0.4 cm Ovary shape: Ovate Ovary color: RHS 139D Fruits/seeds: None observed.
Disease and insect/pest resistance: No special observations were made

### COMPARISON OF WITH PARENTAL LINES AND KNOWN VARIETY

When ‘KLEDG14177’ is compared to the female and male parental lines, the following differences are noted in Table 1:

TABLE 1
Comparison with Parental Lines

| | Variety | Female parent | Male Parent |
|---|---|---|---|
| Trait | ‘KLEDG14177’ | ‘DSS 2009 0513’ | ‘DSS 2008 0003’ |
| Flower color | Dark purple with a dark | Lilac | White |
| | center | | |
| Branching | Very good | Good | Less branching |

When ‘KLEGD14177’ is compared to the commercial line ‘Kahori’ (patent status unknown), the following differences are noted in Table 2:

TABLE 2
Comparison with Similar Variety

| | Variety | Commercial line |
|---|---|---|
| Trait | ‘KLEDG14177’ | ‘Kahori’ |
| Flower color | Dark purple with a dark center | Purple and blue |
| Color of foliage | Dark green to blue | Green |
| Size of leaves | Small | Medium |

We claim:

1. A new and distinct variety of Dianthus plant named ‘KLEDG14177’ as illustrated and described herein.
